(19) United States
(12) Patent Application Publication
(10) Pub. No.: US 2005/0131295 A1
(43) Pub. Date: Jun. 16, 2005

(54) Volumetric ultrasound imaging system using two-dimensional array transducer

(75) Inventor: Xiang-Ning Li, Mill Creek, WA (US)

Correspondence Address: ATL ULTRASOUND, P.O. BOX 3003, 22100 BOTHELL EVERETT HIGHWAY, BOTHELL, WA, 98041-3003, US

(73) Assignee: Koninklijke Philips Electronics N.V.

(21) Appl. No.: 10/985,811

(22) Filed: Nov. 8, 2004

Related U.S. Application Data
(60) Provisional application No. 60/528,797, filed on Dec. 11, 2003.

(51) Int. Cl. A61B 8/00

(52) U.S. Cl. 600/443

(57) ABSTRACT

Volumetric ultrasound images are created using a two-dimensional array transducer to create multiple beams that diverge in an elevational direction and scan in an azimuthal direction. In one embodiment, ultrasound echoes in three beams positioned adjacent each other in the elevational direction are projected onto respective planes. The volumetric image is created by combining the planes of projection for all three beams. The area scanned by the transducer is divided into three beams so that echoes located at the same distance from the transducer are at substantially the same depth beneath the transducer. In another embodiment, multiple beams scan in respective ranges of scanning depths, and the elevational divergence angle is reduced for deeper ranges of scanning depths. As a result, the elevational width of the volumetric image can be relatively constant. In another embodiment, multiple intersecting or parallel beams are used to create volumetric images.

This invention claims the benefit of Provisional U.S. patent Application Ser. No. 60/528,797, filed Dec. 11, 2003.

### TECHNICAL FIELD

This invention relates to ultrasound imaging systems, and, more particularly, to a system and method for performing volumetric imaging using a two-dimensional transducer that scans using multiple fan-shaped beams.

### BACKGROUND OF THE INVENTION

Various noninvasive diagnostic imaging modalities are capable of producing cross-sectional images of organs or vessels inside the body. An imaging modality that is well suited for such real-time noninvasive imaging is ultrasound. Ultrasound diagnostic imaging systems are in widespread use by cardiologists, obstetricians, radiologists and others for examinations of the heart, a developing fetus, internal abdominal organs and other anatomical structures. These systems operate by transmitting waves of ultrasound energy into the body, receiving ultrasound echoes reflected from tissue interfaces upon which the waves impinge, and translating the received echoes into structural representations of portions of the body through which the ultrasound waves are directed.

In conventional ultrasound imaging, objects of interest, such as internal tissues and blood, are scanned using planar ultrasound beams or slices, which are preferably as thin as possible to provide good resolution of such objects accompanied by minimal clutter. A linear array transducer is conventionally used to scan a thin slice by narrowly focusing the transmitted and received ultrasound in an elevational direction and steering the transmitted and received ultrasound throughout a range of angles in an azimuthal direction. A linear array transducer operating in this manner can provide a two-dimensional image representing a cross-section through either a plane that is perpendicular to a face of the transducer for B-mode imaging or parallel to the face of the transducer for C-mode imaging.

Although B-mode and C-mode images are two-dimensional images, it is also possible to generate three-dimensional ultrasound images by either physically moving a linear array or by using a two-dimensional array transducer to steer the transmitted and received ultrasound about two orthogonal axes. Although two-dimensional B-mode or C-mode images can conventionally be generated at a sufficient rate to allow essentially real-time imaging (i.e., at least about 30 frames per second), it is generally not possible at the present time to generate three-dimensional ultrasound images at a rate that is sufficient to permit real-time imaging. Three-dimensional real-time imaging poses two major challenges: first, acquiring echoes from a volume in a sufficiently short time to maintain a real-time image frame rate, and, second, reducing volumetric data obtained from these echoes to a suitable two-dimensional image format with sufficient speed to provide real-time display.

One technique that has been developed to create ultrasound images providing information about anatomical structures in a three-dimensional volume is volumetric imaging, as disclosed in U.S. Pat. No. 5,305,756, which is incorporated herein by reference. Volumetric imaging can generally be accomplished at a sufficient speed to permit real time imaging. With reference to FIG. 1, volumetric imaging is accomplished using a transducer 10 having linear array elements 12. The transmitted and received ultrasound is focused in the azimuthal direction AZ. However, lenses placed on the surface of the elements 12 or the surface geometry of the element 12 themselves cause the ultrasound to diverge in the elevation direction EL to generate a series of fan-shaped beams, collectively shown as 14. The transducer 10 is scanned in a linear array format whereby the ultrasound is sequentially transmitted and received from each array element 12 to form the sequence of fan-shaped beams 14. The beams 14 are orthogonal to the longitudinal surface of the transducer 10 to insonify a volumetric region. In the center of the insonified volumetric region is a plane of projection 18 that bisects each of the fan-shaped beams 14. The plane of projection 18 is spatially represented by the ultrasound image produced by the transducer 10 and is a plane that typically is normal to the surface of the transducer 10 in the azimuthal direction. The resulting ultrasound image provides information about the entire three-dimensional volumetric region because the transducer 10 acoustically integrates all echoes at each range across the entire volumetric region. These echoes are then projected or collapsed onto the plane of projection 18. Since the fan-shaped beams 14 diverge radially in the elevation direction, each constant range locus is a radial line as indicated by a constant range locus 20. Each echo along the constant range locus 20 is projected to a point 22 of intersection of the locus 20 and the plane of projection 18. Since this projection occurs at every range and azimuthal location throughout the volumetric region 16, the image of the plane of projection 18 presents a two-dimensional projection of the entire volume. The resulting image is similar to the two-dimensional projection of a volume obtained using conventional x-ray imaging.

The volumetric image can be obtained as shown in FIG. 1 in essentially real time because all of the echoes at each range across the entire volumetric region isonified by each beam 14 are processed as a single point on the plane of projection 18. As a result, relatively little processing power is required, particularly compared to true three-dimensional ultrasound imaging.

While the transducer 10 may be scanned in a linear array format as shown in FIG. 1 to form a sequence of fan-shaped beams, the transducer 10 may alternatively used by transmitting and receiving properly phased ultrasound signals to and from the array elements 12. By operating the array elements as a phased array, the transducer 10 can electronically steer and focus the ultrasound as shown in FIG. 2. The ultrasound is therefore transmitted and received in a fan-shaped beam 30 that diverges in both the elevational and azimuthal directions. The electronic steering of the beam 30 enable the isonification of a pyramidal shaped volumetric region adjacent the transducer 10. Ultrasound echoes from within this volumetric region are projected onto a triangular shaped plane of projection 36 and used to display a volumetric image.

FIG. 3 illustrates another technique that is described in U.S. Pat. No. 5,305,756 to produce of a fan-shaped beam in the elevational direction. As shown in FIG. 3, a transducer 40 has array elements 42 arranged in two dimensions. As in the transducer 10 of FIGS. 1 and 2, the array elements 42 are aligned in the azimuthal direction. However, each array element 42 is sub-diced in the elevational direction to form sub-elements 46a,b,c. The sub-elements 46a,b,c aligned in the elevational direction allows a series of fan-shaped beams 48 that diverge in the elevational direction to be electronically generated rather than relying upon lenses or the geometry of the element surface to generate a fan-shaped beam. The sub-elements 46a,b,c generate the fan-shaped beams 48 by controlling the time that signals are sent to or received from the sub-elements 46a,b,c. For example, the sub-element 46b could be actuated first, followed in rapid succession by the simultaneous actuation of the sub-elements 46a and 46c. However, it is important to note that the sub-elements 46a,b,c are not used as a phased array in which properly phased ultrasound signals are transmitted from and received by the sub-elements 46a,b,c. Thus, the beams 48 are not steered in the elevational direction. As with the previously described embodiments, the ultrasound echoes in the volumetric region isonified by the beams 48 are projected onto a plane 49 from which the volumetric image is created.

Although the conventional volumetric imaging technique described above represents a significant advance because it allows real time imaging of a three-dimensional volumetric space, it is not without its limitations. For example, as illustrated in FIG. 4A, a transducer 50 shown when viewed in the azimuthal direction scans using a diverging beam 52 as illustrated in FIGS. 1-3. When the transducer 50 is scanning to a range of distances 56 from the transducer 50, all of the points at that range 56 from the transducer 50 will be projected onto a plane of projection 60 as a set of points within a range of depths 62. Therefore, all of the points in that range of distances 56 from the transducer 50 will appear to be in the range of depths 62 on the projection 60 even though the actual depths of the points vary throughout a substantially larger range 66. As a result, viewed in the elevational direction as shown in FIG. 4B, a set of points in the range of depths 62 will be erroneously projected to be within the range of depths 66. Conversely, an anatomical structure that spans a range of depths can appear to be at a single depth because it is a constant distance from the transducer 50.

The problem exemplified by FIGS. 4A, 4B is exacerbated when the elevational divergence angle of the beam 52 is large. Under such circumstances, the volumetric image can fail to clearly show the true configuration of anatomical structures.

Another problem with the conventional three-dimensional volumetric imaging technique shown in FIGS. 1-3 can be explained with reference to FIG. 5. FIG. 5 shows a transducer 80 viewed in the azimuthal direction that is transmitting a beam 82 that diverges in the elevational direction, in the same manner as shown in FIGS. 1-3. The diverging nature of the beam 82 inherently means that the beam 82 will isonify an area of interest beneath the transducer 80 that varies from a relatively small width near the transducer 80 to a relatively large width away from the transducer 80. For example, the beam 82 will isonify a width W₁ at a distance D₁ from the transducer 80, and will isonify a width W₂ at a distance D₂ from the transducer 80. Therefore, the resulting volumetric image will be relatively narrow and show relatively little at the top of the image and will be relatively wide and show substantially more at the bottom of the image. The width of the image can be made equal by cropping the image, such as along lines 86, 88, but doing so wastes image information that would otherwise be viewable.

Still another potential problem that may be encountered in using the three-dimensional volumetric imaging technique shown in FIGS. 1-3 is that certain regions of the image may not be shown in the image with sufficient clarity. For example, since the image does not resolve anatomical structures that lie along the same constant range locus from the transducer, a structure that occupies only a small portion of the constant range locus may be obscured by other anatomical structures that also lie on the constant range locus.

There is therefore a need for a volumetric imaging system and method that clearly shows anatomical structures being imaged without geometric distortion, and does so in a manner that can generate an image having a substantially constant width throughout a range of depths.

### SUMMARY OF THE INVENTION

A system and method of producing volumetric ultrasound images uses a two-dimensional array transducer to scan a region of interest. According to one aspect of the invention, the two-dimensional array transducer scans the region of interest in an azimuthal direction using a plurality of beams that diverge in an elevational direction and are positioned adjacent each other in the elevational direction. Ultrasound reflections in each beam are projection onto a respective plane of projection, and a volumetric ultrasound image is then created by combining the projections on the planes of projection for all of the beams into a common plane of projection.

According to another aspect of the invention, the two-dimensional array transducer scans the region of interest in an azimuthal direction using a plurality of beams that have a common center axis. The beams diverge in an elevational direction in respective divergence angles that are different for each beam. The beams scan respective ranges of scanning depths that are ordered inversely to an order of divergence angles of the beams. As a result, a beam scanning the shallowest range of scanning depths has the largest divergence angle and a beam scanning the deepest range of scanning depths has the smallest divergence angle. The ultrasound reflections in each beam are projected onto a common plane of projection, and the volumetric ultrasound image is created from the ultrasound reflections projected onto the common plane of projection for all of the beams.

In still another aspect of the invention, the two-dimensional array transducer scans the region of interest in an azimuthal direction using a pair of beams. A first beam diverges in a first direction and is used to scan the region of interest in a second direction that is perpendicular to the first direction. Similarly, a second beam diverges in a third direction and is used to scan the region of interest in a fourth direction that is perpendicular to the third direction. Ultrasound reflections in the first beam are projected onto a plane of projection that is perpendicular to the first direction, and ultrasound reflections in the second beam are projected onto a plane of projection that is perpendicular to the third direction. A volumetric ultrasound image is then created from the first and second planes of projection.

### BRIEF DESCRIPTION OF THE DRAWINGS

FIG. 1 is a schematic isometric view illustrating one conventional technique for generating volumetric images.

FIG. 2 is a schematic isometric view illustrating another conventional technique for generating volumetric images.

FIG. 3 is a schematic isometric view illustrating still another conventional technique for generating volumetric images.

FIGS. 4A and 4B are schematic elevational and azimuthal cross-section views, respectively, illustrating a limitation of the conventional volumetric imaging techniques shown in FIGS. 1-3.

FIG. 5 is a schematic elevational cross-section view illustrating another limitation of the conventional volumetric imaging techniques shown in FIGS. 1-3.

FIGS. 6A and 6B are schematic elevational and azimuthal cross-section views, respectively, illustrating a technique for generating volumetric images according to one embodiment of the invention.

FIG. 7 is a schematic elevational cross-section view illustrating a technique for generating volumetric images according to another embodiment of the invention.

FIGS. 8A, 8B, 8C and 8D are schematic views illustrating techniques for generating volumetric images according to still another embodiment of the invention.

FIG. 9 is a block diagram of an ultrasound imaging system that can be used to perform volumetric imaging according to the embodiments shown in FIGS. 6-8.

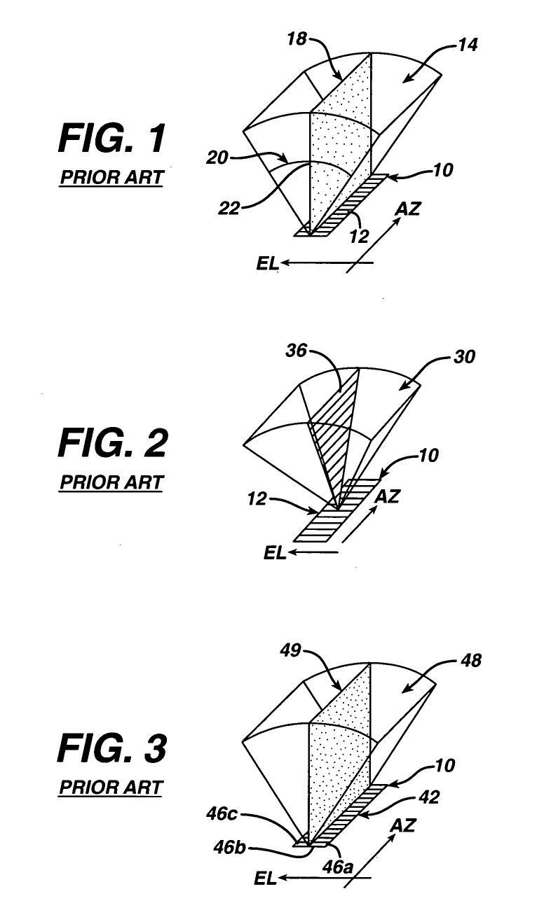

### DETAILED DESCRIPTION

One aspect of the present invention and will now be explained with reference to FIGS. 6A and 6B, which shows views of a two-dimensional array transducer 100 viewed in the azimuthal and elevational directions, respectively. As shown in FIG. 6A, the transducer 100 scans using a diverging center beam 102 and a separate pair of diverging side beams 104, 106. Ultrasound echoes scanned by each of these beams 102, 104, 106 are projected onto respective planes of projection 112, 114, 116. Points at corresponding depths in the planes of projection are then combined to create a single plane of projection that is used to create the volumetric image. The plane of projection 112 can be used as the single plane of projection by transferring points on the planes of projection 114, 116 to the plane of projection 112 at the corresponding depth.

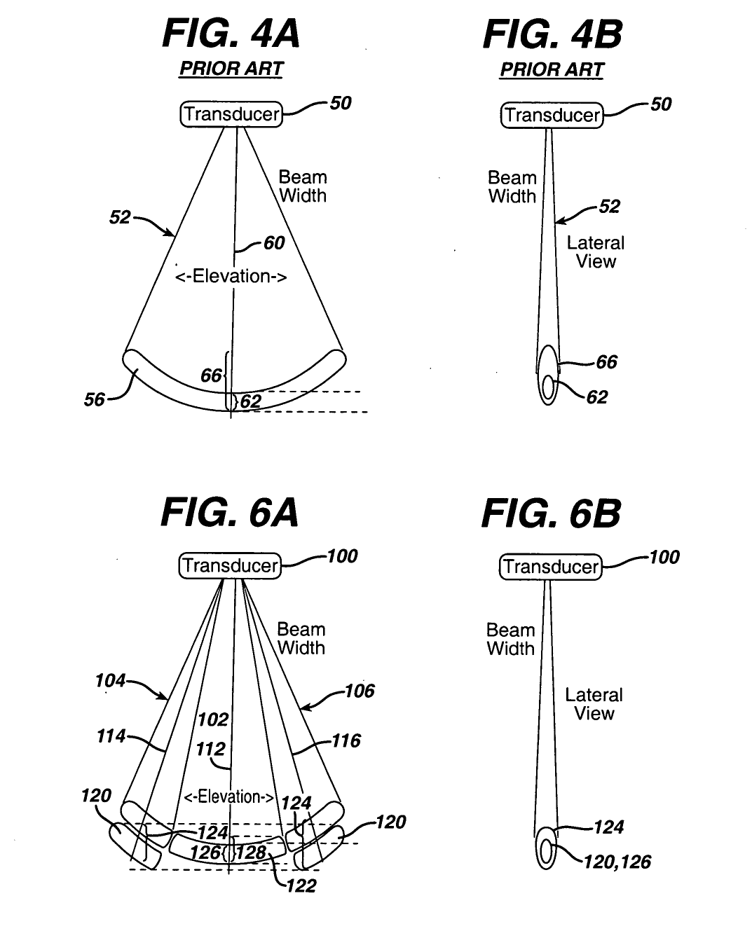

Significantly, the side beams 104, 106 scan to a ranges of distances 120 from the transducer 100 that is greater than a ranges of distances 122 that is scanned using the center beam 102. The difference between the scan distance of the center beam 102 and the scan distance of the side beams 104, 106 is selected so that both scan distances are at substantially the same depth beneath the transducer 100. As a result, the side beams 104, 106 and the center beam 102 scan to substantially the same depth. More specifically, as shown in FIG. 6A, when the transducer 100 causes the center beam 102 to scan in the range of distances 122 from the transducer 100, all of the points in that range of distances 122 will be projected onto the plane of projection 112 within a range of depths 126 that is only slightly smaller than the actual range of depths 128. At the same time, when the transducer 100 causes the side beams 104, 106 to scan at the range of distances 120 from the transducer 100, all of the points in that range 120 will be projected onto the planes of projection 114, 116 as points falling within the range although the actual locations of the points are in a range of depths 124. However, this range of depths 124 differs from the range of distances at which points are projected onto the planes 114, 116 substantially less than in the conventional technique shown in FIGS. 4A and 4B. As a result, when viewed in the elevational direction as shown in FIG. 6B, the depth of anatomical structures will be correctly viewed with substantially less geometric distortion present using the conventional technique shown in FIGS. 4A and 4B. The advantage of using side beams 104, 106 focused to a greater depth than the center beam 102 will be apparent by comparing FIG. 6B with FIG. 4B.

Although the embodiment shown in FIGS. 6A and 6B uses only two side beams 104, 106, it will be understood that a larger number of side beams could be used. Using a larger number of side beams further reduces the geometric distortion that would otherwise be present, but it increases the processing that is required to display an image and may therefore preclude real-time volumetric imaging. Alternatively, volumetric imaging could be accomplished using two side-by-side diverging beams (not shown), but doing so would result in greater geometric distortion but less processing compared to the technique shown in FIGS. 6A and 6B. In general, scanning over a wider area or obtaining an image of greater clarity makes it desirable to use a larger number of beams, particularly if the processing power is available. Regardless of the number of beams that are used, the points on each plane of projection 112, 114, 116 are preferably projected onto a single plane of projection with a weight corresponding to the width of the respective beam. As a result, each ultrasound echo will be projected onto the plane of projection with the same weight regardless of the beam 102-106 that obtained the echo.

The diverging beams 102, 104, 106 can be generated by the two-dimensional transducer 100 using a variety of techniques. The beams 102-106 can be generated by operating array elements of the transducer 100 in a phase-arrayed manner either in respective sub-arrays to form the beams 102-106 at the same time or using all of the array elements of the transducer 100 to sequentially form each individual beam 102-106 at different times. Also, the array elements can be arranged in sub-arrays, each of which is provided with a lens or other mechanical structure to cause a respective beam 102-106 to be generated from the sub-arrays.

One embodiment of another aspect of the present invention is illustrated in FIG. 7, which shows a two-dimensional array transducer 140 that transmits and receives ultrasound and a plurality of sequentially generated beams 142, 144, 146 for scanning within a respective range of depths. The angle of divergence of each beam 142-146 is inversely related to the depth of its scanning range. Thus, the angle of divergence of the beam 142, which scans to a relatively shallow depth, is relatively wide, and the angle of divergence of the beam 146, which scans to a relatively large depth, is relatively narrow. As a result, the width of each beam 142-146 at the furthest extent of its scan depth is substantially the same for all beams 142-146.

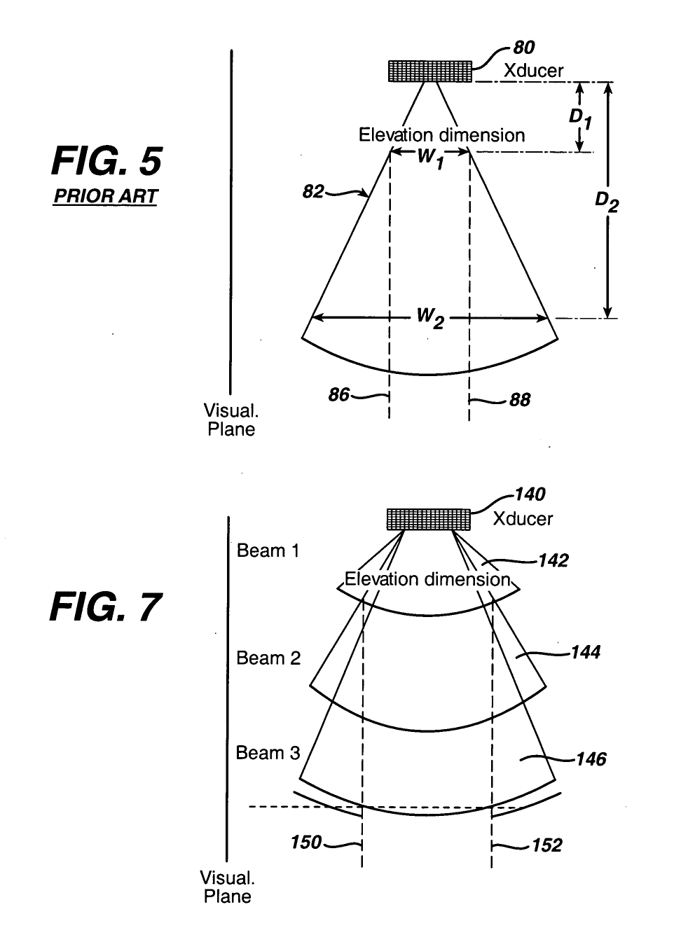

After ultrasound echoes have been obtained using the beams 142-146, a volumetric image is generated by using the echoes within the scan range of each beam 142-146. Thus, the image is generated from relatively shallow echoes using the beam 142, moderately deep echoes using the beam 144, and relatively deep echoes using the beam 146. The resulting image can encompass a width shown by the dotted lines 150, 152, which has a substantially larger width than the image area encompassed by the cropping lines 86, 88 shown in FIG. 5.

A variety of techniques can be used to generate the beams 142-146 with differing divergence angles. However, the beams 142-146 are preferably generated by controlling the array elements of the transducer 140 using phased-array techniques.

The technique shown in FIG. 7 can, of course, be used with a single beam scanning within the each range, or multiple beams can be used to scan within each range using the technique shown in FIGS. 6A and 6B.

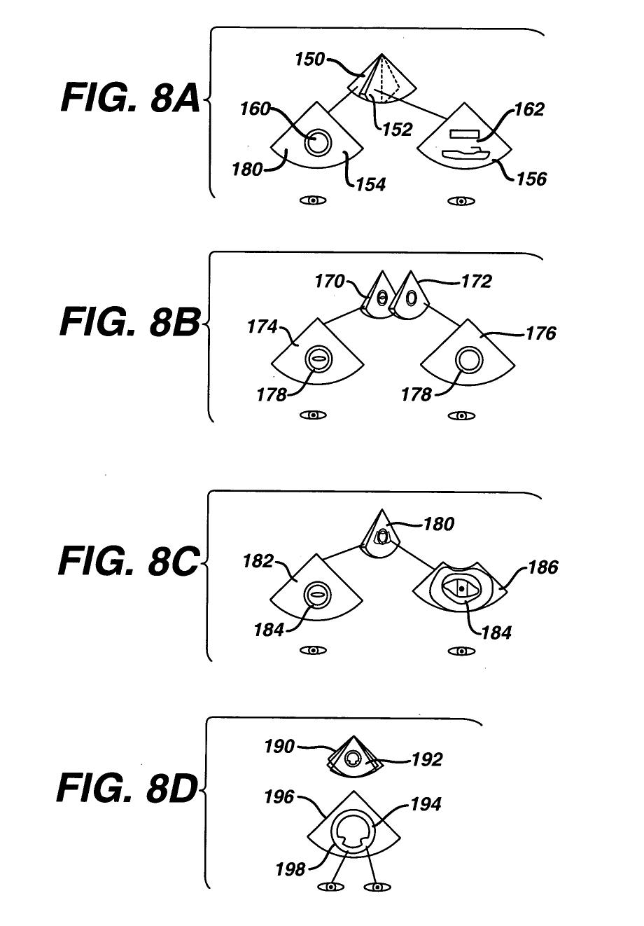

One embodiment of still another aspect of the invention is shown in FIGS. 8A-8D. In this embodiment, the two-dimensional array elements of a transducer (not shown) are used to scan in relatively narrow beams in which all of the points at each range are projected onto a central plane of projection. For example, as shown in FIG. 8A, one volumetric scanning beam 150 is used that is perpendicular to a second volumetric scanning beam 152. The resulting projections 154, 156, respectively, show a vessel in transverse cross-section 160 and longitudinal cross-section 162, respectively.

As shown in FIG. 8B, two parallel scanning beams 170, 172 may be used to generate respective transverse cross sectional projections 174, 176 of a volumetric region of a vessel 178 that are parallel to each other and spaced apart a predetermined distance.

Although the scaling of the projections 154, 156 and 174, 176 is uniform in the embodiments of FIGS. 8A and 8B, volumetric projections of an anatomical structure obtained using the same volumetric scanning beam may be shown with two different degrees of scaling, as shown in FIG. 8C more specifically, a single volumetric scanning beam 180 is used to generate a first projection 182 showing a vessel 184 to actual scale and a second projection 186 showing the vessel 184 in expanded form. This embodiment can allow anatomical structures to be shown with greater clarity.

Finally, FIG. 8D shows two volumetric scanning beams 190, 192 intersecting each other at substantially the same angle that an anatomical structure 194 would be viewed by respective eyes. The beams 190, 192 are used to generate a pair of image projections 196, 198 of the anatomical structure 194, which are viewed by respective eyes so that the depth features of the anatomical structure can be visualized.

Although volumetric scanning beams having a variety of specific geometric relationships have been illustrated in FIGS. 8A-8D, it will be understood that the use of a two-dimensional array transducer allows a great deal of flexibility in the geometric relationships of scanning beams that can be formed. Further, although FIGS. 8A-8D show only one or two volumetric scanning beams being used, it will be understood that a greater number of volumetric scanning beams can be used to create a correspondingly greater number of projected images.

One potential limitation of the various embodiments of the inventive volumetric scanning techniques may be the lack of resolution achievable at a specific depth. As mentioned above, all of the anatomical structures at the same depth are projected onto the same area of a plane of projection. Therefore, an anatomical structure occupying a relatively small width of the scanning beam may be masked or otherwise obscured by other anatomical structures at that same depth. To alleviate this potential problem, three-dimensional scanning can be used to resolve specific anatomical structures. The resulting image of such structures can be overlaid onto the volumetric image. Significantly, the relatively little amount of processing power required to perform volumetric scanning in accordance with the various embodiments of the invention may leave processing power available to perform three-dimensional scanning of limited areas without reducing the acquisition frame rate significantly. As a result, real-time imaging can still be achieved with this limited amount of three-dimensional scanning to overlay volumetric scanning of a larger area.

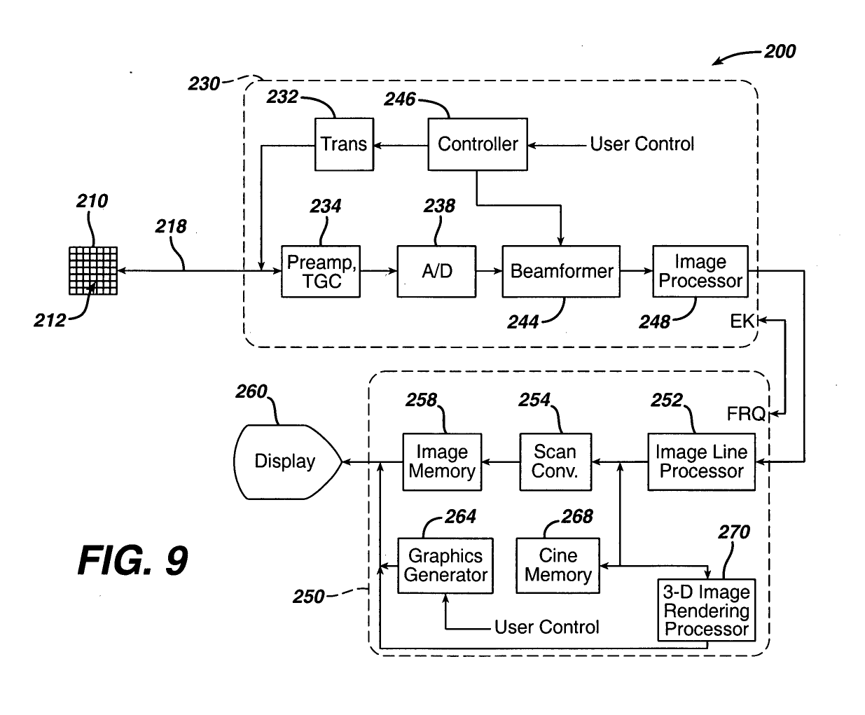

One embodiment of an ultrasound imaging system 200 that can be used to perform volumetric imaging in accordance with the present invention is shown FIG. 9. The imaging system includes a probe 210 having a two-dimensional array of transducer elements 212. The probe 210 is coupled to through a cable 218 to a scanner 230.

The scanner 230 includes a transmitter 232, which generates high frequency signals that are applied to the transducer elements 212 to cause the transducer elements 212 to transmit ultrasound into tissues or blood. Ultrasound echoes of the transmitted ultrasound are received by the transducer elements 212, which generate corresponding analog signals. These analog signals are applied to a preamplifier 234, which amplifies the analog signals. The preamplifier 234 also includes internal TGC (time gain control) circuitry to compensate for attenuation of the transmitted and received ultrasound at greater depths. The amplified and depth compensated signals from the preamplifier 234 are applied to an analog-to-digital (A/D) converter 238 where they are digitized. The digitized echo signals are then formed into beams by a beamformer 244. The beamformer 244 is controlled by a controller 246, which is responsive to a user control. The controller 246 provides control signals to the transmitter 232 instructing the probe 210 as to the timing, frequency, direction and focusing of transmit beams. The controller 246 also controls the beamforming of the digitized echo signals received by the beamformer 244. The output of the beamformer 244 is applied to an image processor 248, which performs digital filtering, B mode detection, and Doppler processing on the beamformed digital signals. The image processor 248 can also perform other signal processing such as harmonic separation, speckle reduction through frequency compounding, and other desired image processing.

Scanning to produce the volumetric images as explained with reference to FIGS. 6-8 is accomplished by the controller 246 controlling the beamformer 244 so that it scans ultrasound echoes having the configurations of the beams shown in FIGS. 6-8. The controller 246 may also control the transmitter 232 so that it transmits ultrasound in beams having the configuration shown in FIGS. 6-8. Since the two-dimensional array of transducer elements 214 has the ability to steer transmitted and received beams in any direction and at any inclination in front of the transducer 212, the beams can have any orientation with respect to the transducer 212 and to each other.

The echo signals produced by the scanner 230 are coupled to the digital display subsystem 250, which processes the echo signals for display in the desired image format. The digital display system 250 includes an image line processor 252, which is samples the echo signals and splices segments of beams into complete line signals. The image line processor also averages line signals for signal-to-noise improvement or flow persistence. The image line signals from the image line processor 252 are applied to a scan converter 254, where they are converted into the desired image format. For example, the scan converter 254 may perform Rho-theta conversion as is known in the art. The image is then stored in an image memory 258 from which it can be displayed on a display 260. The image in the image memory 258 may also be overlaid with graphics to be displayed with the image. The graphics are generated by a graphics generator 264, which is responsive to a user control. Individual images or image sequences can be stored in a cine memory 268 during capture of image loops.

For real-time volumetric imaging, the display subsystem 250 also includes a three-dimensional image rendering processor 270, which receives image lines from the image line processor 252. The three-dimensional image rendering processor 270 renders of a real-time three dimensional image, which is displayed on the display 260.

Although the present invention has been described with reference to preferred embodiments, persons skilled in the art will recognize that changes may be made in form and detail without departing from the spirit and scope of the invention.

1. A method of producing a volumetric ultrasound image, comprising:
using a two-dimensional array transducer to scan a region of interest in an azimuthal direction using a plurality of beams that diverge in an elevational direction, the beams being positioned adjacent each other in the elevational direction;
projecting ultrasound reflections in each beam onto a plane of projection, the reflections being obtained in a range of distances from the transducer and being projected onto the plane of projection at corresponding distances from the transducer; and
creating the volumetric ultrasound image by combining the projections on the plane of projection for each beam into a common plane of projection.

2. The method of claim 1 wherein the act of using a two-dimensional array transducer to scan a region of interest in an azimuthal direction using a plurality of beams that diverge in an elevational direction comprises using each of a plurality of array elements aligned in the azimuthal direction to sequentially scan successive sub-regions region of interest extending in the azimuthal direction.

3. The method of claim 1 wherein the act of using a two-dimensional array transducer to scan a region of interest in an azimuthal direction comprises using each of a plurality of array elements aligned in the azimuthal direction to sequentially scan successive sub-regions extending in the azimuthal direction in the region of interest.

4. The method of claim 1 wherein the act of using a two-dimensional array transducer to scan a region of interest in an azimuthal direction comprises using a plurality of array elements in a phased-array manner to steer each of the beams through a range of angles extending in the azimuthal direction.

5. The method of claim 1 wherein the act of using a two-dimensional array transducer to scan a region of interest in an azimuthal direction using a plurality of beams that diverge in an elevational direction comprises using a center beam positioned between two side beams.

6. The method of claim 1 wherein each of the beams scans a plurality of ranges of scanning depths using respective divergence angles that are ordered inversely to the ranges of scanning depths so that when each of the beams scans its shallowest range of scanning depths it has the largest divergence angle and when each of the beams scans its deepest range of scanning depths it has the smallest divergence angle.

7. The method of claim 1 wherein the volumetric ultrasound image is created in real time.

8. The method of claim 1, further comprising:
using the two-dimensional array transducer to perform a three-dimensional scan of a portion of the region of interest;
creating a three-dimensional ultrasound image from the three-dimensional scan; and
overlaying the three-dimensional ultrasound image on the volumetric ultrasound image.

9. A method of producing a volumetric ultrasound image, comprising:
using a two-dimensional array transducer to scan a region of interest in an azimuthal direction using a plurality of beams that have a common center axis, the beams diverging in an elevational direction in respective divergence angles that are different for each beam, the beams scanning respective ranges of scanning depths that are ordered inversely to an order of divergence angles of the beams so that a beam scanning the shallowest range of scanning depths has the largest divergence angle and a beam scanning the deepest range of scanning depths has the smallest divergence angle;
projecting ultrasound reflections in each beam onto a common plane of projection, the reflections obtained for each beam being in the respective range of scanning depth; and
creating the volumetric ultrasound image from the ultrasound reflections projected onto the common plane of projection for all of the beams.

10. The method of claim 9 wherein all of the beams have substantially the same dimension in the elevational direction at the maximum depth in their respective ranges of scanning depths.

11. The method of claim 9 wherein the volumetric ultrasound image is created in real time.

12. The method of claim 9, further comprising:
using the two-dimensional array transducer to perform a three-dimensional scan of a portion of the region of interest;
creating a three-dimensional ultrasound image from the three-dimensional scan; and
overlaying the three-dimensional ultrasound image on the volumetric ultrasound image.

13. A method of producing a volumetric ultrasound image, comprising:
using a two-dimensional array transducer to scan a region of interest in an azimuthal direction using a beam that diverges in an elevational direction, the beam scanning a plurality of ranges of scanning depths using respective divergence angles that are ordered inversely to the ranges of scanning depths so that when the beam scans the shallowest range of scanning depths it has the largest divergence angle and when the beam scans the deepest range of scanning depths it has the smallest divergence angle;
projecting ultrasound reflections at each range of scanning depths onto a plane of projection; and
creating the volumetric ultrasound image from the ultrasound reflections projected onto the plane of projection.

14. The method of claim 13 wherein the beam has substantially the same dimension in the elevational direction at the maximum depth in each of the ranges of scanning depths.

15. The method of claim 13 wherein the volumetric ultrasound image is created in real time.

16. The method of claim 13, further comprising:
using the two-dimensional array transducer to perform a three-dimensional scan of a portion of the region of interest;
creating a three-dimensional ultrasound image from the three-dimensional scan; and
overlaying the three-dimensional ultrasound image on the volumetric ultrasound image.

17. A method of producing a volumetric ultrasound image, comprising:
using a two-dimensional array transducer to scan a region of interest using a pair of beams, a first of the beams diverging in a first direction and being used to scan the region of interest in a second direction that is perpendicular to the first direction, a second of the beams diverging in a third direction and being used to scan the region of interest in a fourth direction that is perpendicular to the third direction;
projecting ultrasound reflections in the first beam onto a plane of projection that is perpendicular to the first direction;
projecting ultrasound reflections in the second beam onto a plane of projection that is perpendicular to the third direction; and
creating the volumetric ultrasound image from the first and second planes of projection.

18. The method of claim 17 wherein the second direction is parallel to the third direction so that the first and second planes of projection are parallel to each other.

19. The method of claim 17 wherein the second direction is perpendicular to the third direction so that the first and second planes of projection intersect each other at a right angle.

20. The method of claim 17 wherein the volumetric ultrasound image is created in real time.

21. An ultrasound diagnostic imaging system comprising:
a two-dimensional array transducer;
a beamformer coupled to the two-dimensional array transducer to beamform received ultrasound echo signals;
a controller coupled to the two-dimensional array transducer, the controller controlling the two-dimensional array transducer to scan a region of interest in an azimuthal direction using a plurality of beams that diverge in an elevational direction, the beams being positioned adjacent each other in the elevational direction;
a processor processing the beamformed ultrasound echo signals and projecting ultrasound echoes scanned by each beam onto a respective plane of projection; and
a display subsystem coupled to the processor, the display subsystem creating a volumetric ultrasound image by combining the projections on the plane of projection for each beam into a common plane of projection.

22. The system of claim 21 wherein the controller controls the two-dimensional array transducer to scan a region of interest in an azimuthal direction by using each of a plurality of array elements in the two dimensional array transducer that are aligned in the azimuthal direction to sequentially scan successive sub-regions region of interest extending in the azimuthal direction.

23. The system of claim 21 wherein the controller controls the two dimensional array transducer to scan a region of interest in an azimuthal direction by using a plurality of array elements in the two-dimensional array transducer in a phased-array manner to steer each of the beams through a range of angles extending in the azimuthal direction.

24. The system of claim 21 wherein the controller controls the two dimensional array transducer so that each of the beams scans a plurality of ranges of scanning depths using respective divergence angles that are ordered inversely to the ranges of scanning depths so that when each of the beams scans its shallowest range of scanning depths it has the largest divergence angle and when each of the beams scans its deepest range of scanning depths it has the smallest divergence angle.

25. The system of claim 21 wherein the volumetric ultrasound image is created in real time.

26. An ultrasound diagnostic imaging system comprising:
a two-dimensional array transducer;
a beamformer coupled to the two-dimensional array transducer to beamform received ultrasound echo signals;
a controller coupled to the two-dimensional array transducer, the controller controlling the two-dimensional array transducer to scan a region of interest in an azimuthal direction using a plurality of beams that have a common center axis, the beams diverging in an elevational direction in respective divergence angles that are different for each beam, the controller causing the beams to scan respective ranges of scanning depths that are ordered inversely to an order of divergence angles of the beams so that a beam scanning the shallowest range of scanning depths has the largest divergence angle and a beam scanning the deepest range of scanning depths has the smallest divergence angle;
a processor processing the beamformed ultrasound echo signals and projecting ultrasound echoes scanned by each beam onto a common plane of projection, the ultrasound echoes scanned by each beam being in the respective range of scanning depth; and
a display subsystem coupled to the processor, the display subsystem creating a volumetric ultrasound image from the ultrasound echoes projected onto the plane of projection for all of the beams.

27. The system of claim 26 wherein the controller controls the two-dimensional array transducer so that all of the beams have substantially the same dimension in the elevational direction at the maximum depth in their respective ranges of scanning depths.

28. The system of claim 26 wherein the volumetric ultrasound image is created in real time.

29. An ultrasound diagnostic imaging system comprising:
a two-dimensional array transducer;
a beamformer coupled to the two-dimensional array transducer to beamform received ultrasound echo signals;
a controller coupled to the two-dimensional array transducer, the controller controlling the two-dimensional array transducer to scan a region of interest using a pair of beams, a first of the beams diverging in a first direction and being used to scan the region of interest in a second direction that is perpendicular to the first direction, a second of the beams diverging in a third direction and being used to scan the region of interest in a fourth direction that is perpendicular to the third direction;
a processor processing the beamformed ultrasound echo signals and projecting ultrasound echoes scanned by the first beam onto a plane of projection that is perpendicular to the first direction and projecting ultrasound echoes scanned by the second beam onto a plane of projection that is perpendicular to the third direction;
a display subsystem coupled to the processor, the display subsystem creating a volumetric ultrasound image from the first and second planes of projection.

30. The system of claim 29 wherein the second direction is parallel to the third direction so that the first and second planes of projection are parallel to each other.

31. The system of claim 29 wherein the second direction is perpendicular to the third direction so that the first and second planes of projection intersect each other at a right angle.
